(19) United States
(12) Patent Application Publication
(10) Pub. No.: US 2004/0067870 A1
(43) Pub. Date: Apr. 8, 2004

(54) Fragrance raw materials aldehydes and pro-fragrances having a tertiary alpha carbon atom

(75) Inventor: Gregory Scott Miracle, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: THE PROCTER & GAMBLE COMPANY

(21) Appl. No.: 10/678,207

(22) Filed: Oct. 3, 2003

Related U.S. Application Data
(63) Continuation of application No. 10/030,759, filed on Oct. 22, 2001, now abandoned.
Application No. 10/030,759 is a 371 of international application No. PCT/US00/10211, filed on Apr. 14, 2000.
(60) Provisional application No. 60/130,127, filed on Apr. 20, 1999.

(51) Int. Cl. A61K 7/46

(52) U.S. Cl. 512/10; 512/11; 512/26

(57) ABSTRACT

The present invention relates to fragrance raw materials having a tertiary alpha carbon atom, to fragrance delivery systems which comprise said tertiary alpha carbon atom fragrance raw materials, and pro-fragrances which are capable of delivering said tertiary alpha carbon atom fragrance raw material and thereby providing an enhanced and sustained aesthetic fragrance benefit. The compounds and systems of the present invention are suitable for use in fine fragrances, perfumes, and other personal care compositions.

### CROSS REFERENCE TO RELATED APPLICATION

This application is a continuation of U.S. application Ser. No. 10/030,759, which is the National Stage of Internation Application PCT/US00/10211, filed Apr. 14, 2000, which claims the benefit under 35 USC §119(e) of U.S. Provisional Application No. 60/130,127, filed Apr. 20, 1999.

### FIELD OF THE INVENTION

The present invention relates to novel fragrance pro-accords which release fragrance raw material aldehydes suitable for use in perfume and fine fragrance compositions thereby providing said compositions with enduring fragrance characteristics. The present invention further relates to a fragrance delivery system which comprises the novel fragrance pro-accords of the present invention in combination with a) one or more pro-accords inter alia acetals, ketals, orthoesters, orthocarbonates, and b) one or more fragrance raw materials.

### BACKGROUND OF THE INVENTION

Humans have applied scents and fragrances to their skin since antiquity. Originally these aesthetically pleasing materials were commonly isolated in raw form as resins, gums or essential oils from natural sources, inter alia, the bark, roots, leaves and fruit of indigenous plants. These resins, gums, and oils were directly applied to the body or diluted with water or other solvent, including in some cases, wine. With the advent of modern chemistry, individual components responsible for the odor properties of these resins, gums and oils were isolated and subsequently characterized. Aside from common “perfume vehicles” inter alia, fine perfumes, colognes, eau de toilettes, and after-shave lotions, a wide variety of personal care or personal hygiene items also deliver for aesthetic reasons fragrance notes, accords, or fragrance “characteristics”.

It is well known that mixtures of perfume or fragrance raw materials when deposited on the skin lose intensity and may change character with time, mainly due to factors such as differential evaporation and skin penetration. Many attempts have been made to minimize these drawbacks, but so far without notable success. Particularly, efforts have been made to prolong the diffusion, as well as to improve other characteristics of fragrance materials, by e.g. increasing the fragrance raw material concentration or by using additives such as silicones, glycerol, polyethylene glycols and so on. Such additions, however, have never been adequate to increase the longevity of the fragrance odor.

In addition to alcohols, ketones, and esters, aldehydes form the most commonly delivered fragrance raw materials. Alcohols and esters can be suitably released in a delayed manner from an orthoester pro-accord or pro-fragrance. The controllable release of these fragrance raw materials thus provides the formulator with a means for delivering these fragrance ingredients, not only as an accord, but in a delayed-releasable manner over a period of time fragrance which is desirable to the fine fragrance and perfume user. However, the primary means for delivering aldehydes in a time-releasable manner has typically been via the acetal pro-fragrance. Notwithstanding the fact that these materials are capable of delivering the required aldehyde under the proper acidic conditions, in the past, because there was no means of adjusting the release profiles of pro-fragrances, these compounds have not provided the formulator with a highly controllable method for sustained and predictable delivery of aldehydes and ketones.

In addition, there is a need for novel aldehyde fragrance raw materials which have the ability to provide subtle changes to a parent “fragrance” or which will provide a heretofore unknown characteristic to a perfume accord.

Accordingly, there remains a need in the art for novel fragrance or perfume raw materials. In addition, there is a corresponding need for a means of delivering said novel fragrance raw materials via a pro-accord. These pro-accords and the released fragrance raw materials are components of a fragrance delivery system which can be formulated into fine fragrances, perfumes, personal care and personal hygiene products. Once formulated therein, the aldehyde fragrance raw material component can be released in a highly controllable manner to provide and additional enhanced fragrance longevity benefit.

### SUMMARY OF THE INVENTION

A first aspect of the present invention relates to novel fragrance raw material aldehydes which comprise a tertiary α-carbon having at least one alkyl group, preferably a methyl group, said fragrance raw materials having the formula:

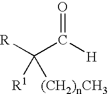

wherein R and R¹ are each independently:

a) C₁-C₁₀ substituted or unsubstituted linear alkyl;

b) C₃-C₁₅ substituted or unsubstituted branched alkyl;

c) C₂-C₁₅ substituted or unsubstituted linear alkenyl;

d) C₃-C₁₅ substituted or unsubstituted branched alkenyl;

e) C₃-C₁₅ substituted or unsubstituted cycloalkyl;

f) C₄-C₁₅ substituted or unsubstituted branched cycloalkyl;

g) C₄-C₁₅ substituted or unsubstituted cycloalkenyl;

h) C₅-C₁₅ substituted or unsubstituted branched cycloalkenyl;

i) C₆-C₁₅ substituted or unsubstituted aryl;

j) C₇-C₂₂ substituted or unsubstituted alkylenearyl;

k) C₇-C₂₂ substituted or unsubstituted arylenealkyl;

l) C₆-C₂₂ substituted or unsubstituted heterocyclicalkyl;

m) C₆-C₂₂ substituted or unsubstituted heterocyclicalkenyl;

n) hydroxyl;

o) nitrilo;

p) a carbonyl comprising unit having the formula:

—(CH₂)ₓCOR²

wherein R² is:

i) —OH;

ii) —OR³ wherein R³ is hydrogen, C₁-C₁₅ substituted or unsubstituted linear alkyl, C₁-C₁₅ substituted or unsubstituted branched alkyl, C₂-C₂₂ substituted or unsubstituted linear alkenyl, C₃-C₂₂ substituted or unsubstituted branched alkenyl, or mixtures thereof;

iii) —N(R⁴)₂ wherein R⁴ is hydrogen, C₁-C₆ substituted or unsubstituted linear alkyl, C₃-C₆ substituted or unsubstituted branched alkyl, or mixtures thereof;

iv) C₁-C₂₂ substituted or unsubstituted linear alkyl;

v) C₁-C₂₂ substituted or unsubstituted branched alkyl;

vi) C₂-C₂₂ substituted or unsubstituted linear alkenyl;

vii) C₃-C₂₂ substituted or unsubstituted branched alkenyl;

viii) C₃-C₂₂ substituted or unsubstituted cycloalkyl;

ix) C₆-C₂₂ substituted or unsubstituted aryl;

x) C₆-C₂₂ substituted or unsubstituted heterocyclicalkyl;

xi) C₆-C₂₂ substituted or unsubstituted heterocyclicalkenyl;

the index x is from 0 to 22;

q) alkyleneoxy units having the formula:

—(CR⁵R⁶)_{y}(CHR⁷CHR⁸O)_{z}R⁹

wherein each R⁵, R⁶, and R⁷ is independently;

i) hydrogen;

ii) —OH;

iii) C₁-C₄ alkyl;

iv) or mixtures thereof;

R⁸ is:

i) hydrogen;

ii) C₁-C₄ alkyl;

iii) or mixtures thereof;

R⁹ is:

i) hydrogen;

ii) C₁-C₄ alkyl;

iii) or mixtures thereof;

R⁵ and R⁶ can be taken together to form a C₃-C₆ spiroannulated ring, carbonyl unit, or mixtures thereof; y has the value from 0 to 10, z has the value from 1 to 50;

r) R and R¹ can be taken together to form:

i) a C₃-C₆ substituted or unsubstituted spiroannulated ring;

ii) a substituted or unsubstituted non-aromatic ring comprising from 5 to 7 atoms in the ring;

iii) a substituted or unsubstituted non-aromatic heterocyclic ring comprising from 5 to 7 atoms in the ring;

iv) a substituted or unsubstituted fused ring system comprising from 5 to 20 atoms; and

n is 0 or 1.

The present invention further relates to fragrance raw material releasing pro-fragrances or pro-accords which comprise the novel aldehydes of the present invention.

The present invention also relates to fragrance raw material delivery systems, said delivery systems comprising:

A) from about 1% by weight, of a pro-fragrance component comprising:

i) at least 1% by weight, of an a-tertiary carbon aldehyde releasing pro-fragrance component:

ii) optionally from about 1% by weight, of one or more pro-accords formed from at least one fragrance raw material, wherein said pro-accord is selected from the group consisting of acetals, ketals, orthoesters, orthocarbonates, and mixtures thereof, each pro-accord releasing upon hydrolysis said fragrance raw material from which it is formed, said fragrance raw materials selected from the group consisting of primary, secondary, and tertiary alcohols, aldehydes, ketones, esters, carbonates, and mixtures thereof, provided each pro-accord:

a) is formed from at least one fragrance raw material having a molecular weight greater than or equal to about 100 g/mol;

b) has a fragrance release half-life of greater than or equal to about 0.1 hours at pH 5.3 and less than or equal to about 12 hours at pH 2.5 when measured in NaH₂PO₄ buffer;

iii) the balance carriers, stabilizers, and other adjunct ingredients; and

B) optionally from about 1% by weight, a fragrance raw material component comprising:

i) optionally at least 0.1% by weight, of an a -tertiary carbon aldehyde according to the present invention;

ii) optionally at least 1% by weight, of a mixture of one or more base note fragrances;

iii) optionally at least 1% by weight, of a mixture of one or more top or middle note fragrances;

iv) optionally the balance carriers, fixatives, and other adjunct ingredients.

These and other objects, features and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference.

### DETAILED DESCRIPTION OF THE INVENTION

The present invention relates to novel aldehyde fragrance raw materials and pro-fragrances which are capable of releasing said aldehyde fragrance raw materials. The novel aldehydes and pro-fragrances comprise a fragrance delivery system which can be suitably formulated into fine fragrances, eau de toilettes, body washes, perfumes, deodorants, and other personal care and personal hygiene articles.

Aldehyde Fragrance Raw Materials

The novel aldehydes of the present invention which function as fragrance raw materials have the general formula:

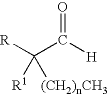

wherein R is independently:

a) C₁-C₁₀ substituted or unsubstituted linear alkyl;

b) C₃-C₁₅ substituted or unsubstituted branched alkyl;

c) C₂-C₁₅ substituted or unsubstituted linear alkenyl;

d) C₃-C₁₅ substituted or unsubstituted branched alkenyl;

e) C₃-C₁₅ substituted or unsubstituted cycloalkyl;

f) C₄-C₁₅ substituted or unsubstituted branched cycloalkyl;

g) C₄-C₁₅ substituted or unsubstituted cycloalkenyl;

h) C₅-C₁₅ substituted or unsubstituted branched cycloalkenyl;

i) C₆-C₁₅ substituted or unsubstituted aryl;

i) C₇-C₂₂ substituted or unsubstituted alkylenearyl;

k) C₇-C₂₂ substituted or unsubstituted arylenealkyl;

l) C₆-C₂₂ substituted or unsubstituted heterocyclicalkyl;

m) C₆-C₂₂ substituted or unsubstituted heterocyclicalkenyl;

n) hydroxyl;

o) nitrilo;

p) a carbonyl comprising unit having the formula:

—(CH₂)ₓCOR²

wherein R² is:

i) —OH;

ii) —OR³ wherein R³is hydrogen, C₁-C₁₅ substituted or unsubstituted linear alkyl, C₁-C₁₅ substituted or unsubstituted branched alkyl, C₂-C₂₂ substituted or unsubstituted linear alkenyl, C₃-C₂₂ substituted or unsubstituted branched alkenyl, or mixtures thereof;

iii) —N(R⁴)₂ wherein R⁴ is hydrogen, C₁-C₆ substituted or unsubstituted linear alkyl, C₃-C₆ substituted or unsubstituted branched alkyl, or mixtures thereof;

iv) C₁-C₂₂ substituted or unsubstituted linear alkyl;

v) C₁-C₂₂ substituted or unsubstituted branched alkyl;

vi) C₂-C₂₂ substituted or unsubstituted linear alkenyl;

vii) C₃-C₂₂ substituted or unsubstituted branched alkenyl;

viii) C₃-C₂₂ substituted or unsubstituted cycloalkyl;

ix) C₆-C₂₂ substituted or unsubstituted aryl;

x) C₆-C₂₂ substituted or unsubstituted heterocyclicalkyl;

xi) C₆-C₂₂ substituted or unsubstituted heterocyclicalkenyl;

the index x is from 0 to 22;

q) alkyleneoxy units having the formula:

—(CR⁵R⁶)_{y}(CHR⁷CHR⁸O)_{z}R⁹

wherein each R⁵, R⁶, and R⁷is independently;

i) hydrogen;

ii) —OH;

iii) C₁-C₄ alkyl;

iv) or mixtures thereof;

R⁸ is:

i) hydrogen;

ii) C₁-C₄ alkyl;

iii) or mixtures thereof;

R⁹ is:

i) hydrogen;

ii) C₁-C₄ alkyl;

iii) or mixtures thereof;

R⁵ and R⁶ can be taken together to form a C₃-C₆ spiroannulated ring, carbonyl unit, or mixtures thereof; y has the value from 0 to 10, z has the value from 1 to 50;

R¹ is independently:

a) C₁-C₁₀ substituted or unsubstituted linear alkyl;

b) C₃-C₁₀ substituted or unsubstituted branched alkyl;

c) C₂-C₁₀ substituted or unsubstituted linear alkenyl;

d) C₃-C₁₀ substituted or unsubstituted branched alkenyl;

e) C₃-C₁₅ substituted or unsubstituted cycloalkyl;

f) C₄-C₁₅ substituted or unsubstituted branched cycloalkyl;

g) C₄-C₁₅ substituted or unsubstituted cycloalkenyl;

h) C₅-C₁₅ substituted or unsubstituted branched cycloalkenyl;

i) C₆-C₁₅ substituted or unsubstituted aryl;

j) C₇-C₂₂ substituted or unsubstituted alkylenearyl;

k) C₇-C₂₂ substituted or unsubstituted arylenealkyl;

l) C₆-C₂₂ substituted or unsubstituted heterocyclicalkyl;

m) C₆-C₂₂ substituted or unsubstituted heterocyclicalkenyl;

n) hydroxyl;

o) nitrilo;

p) a carbonyl comprising unit having the formula:

—(CH₂)ₓCOR²

wherein R² is:

i) —OH;

ii) —OR³ wherein R³is hydrogen, C₁-C₁₅ substituted or unsubstituted linear alkyl, C₁-C₁₅ substituted or unsubstituted branched alkyl, C₂-C₂₂ substituted or unsubstituted linear alkenyl, C₃-C₂₂ substituted or unsubstituted branched alkenyl, or mixtures thereof;

iii) —N(R⁴)₂ wherein R⁴ is hydrogen, C₁-C₆ substituted or unsubstituted linear alkyl, C₃-C₆ substituted or unsubstituted branched alkyl, or mixtures thereof;

iv) C₁-C₂₂ substituted or unsubstituted linear alkyl;

v) C₁-C₂₂ substituted or unsubstituted branched alkyl;

vi) C₂-C₂₂ substituted or unsubstituted linear alkenyl;

vii) C₃-C₂₂ substituted or unsubstituted branched alkenyl;

viii) C₃-C₂₂ substituted or unsubstituted cycloalkyl;

ix) C₆-C₂₂ substituted or unsubstituted aryl;

x) C₆-C₂₂ substituted or unsubstituted heterocyclicalkyl;

xi) C₆-C₂₂ substituted or unsubstituted heterocyclicalkenyl;

the index x is from 0 to 22;

q) alkyleneoxy units having the formula:

—(CR⁵R⁶)_{y}(CHR⁷CHR⁸O)_{z}R⁹

wherein each R⁵, R⁶, and R⁷ is independently;

i) hydrogen;

ii) —OH;

iii) C₁-C₄ alkyl;

iv) or mixtures thereof;

R⁸ is:

i) hydrogen;

ii) C₁-C₄ alkyl;

iii) or mixtures thereof;

R⁹ is:

i) hydrogen;

ii) C₁-C₄ alkyl;

iii) or mixtures thereof;

R⁵ and R⁶ can be taken together to form a C₃-C₆ spiroannulated ring, carbonyl unit, or mixtures thereof; y has the value from 0 to 10, z has the value from 1 to 50;

r) R and R¹ can be taken together to form:

i) a C₃-C₂₀ substituted or unsubstituted spiroannulated ring system;

ii) a substituted or unsubstituted non-aromatic ring system comprising from 5 to 20 atoms; for example a fragrance raw material aldehyde having the formula:

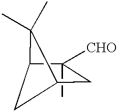

iii) a substituted or unsubstituted non-aromatic heterocyclic ring system comprising from 5 to 7 atoms;

iv) a substituted or unsubstituted fused ring system comprising from 5 to 20 atoms, for example, a fragrance raw material aldehyde having the formula:

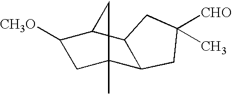

and

n is 0 or 1.

The following are non-limiting examples of preferred fragrance raw material aldehydes according to the present invention:

2,2,6,10-tetramethyl-9-undecenal;

2-ethyl-2,6,10-trimethyl-9-undecenal;

2-ethyl-2-methyldecanal;

2,2-dimethyl-10-undecenal;

2-ethyl-2-methyl-10-undecenal;

2,2-dimethylundecanal;

2-ethyl-2-methylundecanal;

2,2-dimethyldodecanal;

2-ethyl-2-methyldodecanal;

2-ethyl-2-methyloctanal;

2-ethyl-2-methylnonanal;

α-methyl-α-pentylbenzepropanal;

α-ethyl-α-pentylbenzepropanal;

2-ethyl-2,3,7-trimethyl-6-octenal;

α, α.2,6,6-pentamethyl-1-cyclohexene-1-butanal;

α-ethyl-α.2,6,6-tetramethyl-1-cyclohexene-1-butanal;

4-(1,1-dimethylethyl)-α-ethyl-α-methylbenzenepropanal;

4-methoxy-α-ethyl-α-methylbenzenepropanal;

1,2,3,4,5,6,7,8-octahydro-2-ethyl-8,8-dimethyl-2-naphthalenecarboxaldehyde

1,2,3,4,5,6,7,8-octahydro-2-ethyl-5,5-dimethyl-2-naphthalenecarboxaldehyde;

2-methyl-2-(2,6-dimethyl-5-heptenoxy)propanal;

2-ethyl-2-(2,6-dimethyl-5-heptenoxy)propanal;

2-ethyl-2-phenoxypropanal;

7-hydroxy-2,2,3,7-tetramethyloctanal;

7-hydroxy-2-ethyl-2,3,7-trimethyloctanal;

2-ethyl-2-methyl4-decenal;

2,2-dimethyl-8-decenal;

2-ethyl -2-methyl-8-decenal;

α-ethyl-α-methyl-4-(1-methylethyl)benzenepropanal;

2-methyl-[(3,7-dimethyl-2,6-octadienyl)oxy]propanal;

2-ethyl-[(3,7-dimethyl-2,6-octadienyl)oxy]propanal;

α-ethyl-α-methyl-1,3-benzodioxole-5-propanal;

octahydro-4,7-methano-1H-indene-5-(2,2-dimethyl)butanal;

octahydro-4,7-methano-1H-indene-5-(2-ethyl-2-methyl)butanal;

α-ethyl-α-methyl-4-(1-methylethyl)benzeneacetaldehyde;

7-hydroxy-2,2,3,7-tetramethyloctanal;

7-hydroxy-2-ethyl-2,3,7-trimethyloctanal;

1,2,4,6-tetramethyl-3-cyclohexene-1-carboxaldehyde;

1-ethyl-2,4,6-trimethyl-3-cyclohexene-1-carboxaldehyde;

α-hexyl-α-methylbenzenepropanal;

α-ethyl-α-hexylbenzenepropanal;

2-ethyl-2-methyl-3-dodecenal;

4-(4-hydroxy-4-methylpentyl)-1-ethyl-3-cyclohexene-1-carboxaldehyde;

3-(4-hydroxy-4-methylpentyl)-1-methyl-3-cyclohexene-1-carboxaldehyde;

3-(4-hydroxy-4-methylpentyl)-1-ethyl-3-cyclohexene-1-carboxaldehyde;

7-methoxy-2,2,3,7-tetramethyloctanal;

7-methoxy-2-ethyl-2,3,7-trimethyloctanal;

αα.β-trimethylbenzenepentanal;

α.β-dimethyl-α-ethylbenzenepentanal;

α-ethyl-α-methylbenzenepropanal;

2-ethyl-2,6-dimethyl-5-heptenal;

2,2-dimethyl-6-nonenal;

2-ethyl-2-methyl-6-nonenal;

α-ethyl-α,4-dimethylbenzeneacetaldehyde;

1,3,5,6-tetramethyl-3-cyclohexene-1-carboxaldehyde;

1-ethyl-3,5,6-trimethyl-3-cyclohexene-1-carboxaldehyde;

2,2,6,10-tetramethyl-5,9-undecadienal;

2-ethyl-2,6,10-trimethyl-5,9-undecadienal;

5,5-dimethyl-2-(2-methyl-2-carboxaldehydepropyl)bicyclo[2.1.1]hex-2-ene;

5,5-dimethyl-2-(2-methyl-2-carboxaldehydebutyl)bicyclo[2.1.1]hex-2-ene; octahydro-3-methyl-5-methoxy-4,7-methano-1H-indene-3-carboxaldehyde; octahydro-3-ethyl-5-methoxy-4,7-methano-1H-indene-3-carboxaldehyde; octahydro-2-methyl-5-methoxy-4,7-methano-1H-indene-2-carboxaldehyde; octahydro-2-ethyl-5-methoxy-4,7-methano-1H-indene-2-carboxaldehyde; octahydro-1-methyl-5-methoxy-4,7-methano-1H-indene-1-carboxaldehyde; octahydro-2-ethyl-5-methoxy-4,7-methano-1H-indene-2-carboxaldehyde; octahydro-4,7-Methano-1H-indene-2-methyl-2-carboxaldehyde; octahydro-4,7-Methano-1H-indene-2-ethyl-2-carboxaldehyde;

2,2,3,7-tetramethyloctanal; 2-ethyl-2,3,7-trimethyloctanal;

α-ethyl-α,β-dimethylbenzenepropanal;

octahydro-5-methyl-4,7-methano-1H-indene-5-carboxaldehyde;

octahydro-5-ethyl-4,7-methano-1H-indene-5-carboxaldehyde;

2-ethyl-2-(4-methylphenoxy)propanal;

2,2,3,5,5-pentamethylhexanal;

2-ethyl-2,3,5,5-tetramethylhexanal;

2-methyl-3-propylbicyclo[2.2.1]hept-5-ene-2-carboxaldehyde;

2-ethyl-3-propylbicyclo[2.2.1]hept-5-ene-2-carboxaldehyde.

Aldehyde Pro-Fragrances

The present invention further relates to pro-fragrances which release the aldehydes of the present invention. The pro-fragrances are suitable for use in the fragrance delivery system which is further described herein below. Key to the aldehyde releasing pro-fragrances of the present invention is the ability of the formulator to prepare a compound pro-fragrance which has a specific release rate of aldehyde fragrance raw material. The formulator may design pro-fragrances to release at any rate which the formulator finds useful to the consumer. In addition, by determining the Fragrance Release Half-life of each particular pro-fragrance, the formulator is able to make a side by side comparison of structurally dissimilar molecules and evaluate their relative fragrance raw material release patterns. In this way not only can the formulator determine with minimal experimentation whether a compound is suitable for use as a pro-fragrance, but also determine the release profile of the molecule. Therefore, a slight structural modification can result in a more finely tuned fragrance delivery system.

The aldehyde releasing pro-fragrances of the present invention are preferably preferaby oxazolidines, tertahydro-1,3-oxazines, thiazolidines, or tetrahydro-1,3-thiazines, more preferably oxazolidines, or tertahydro-1,3-oxazines, most preferably oxazolidines having the formula:

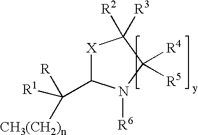

wherein R, R¹, and n are the same as defined herein above X is oxygen or sulfur, each R², R³, R⁶ and each R⁴ and R⁵ pair are independently:

a) hydrogen;

b) R;

c) hydroxyl;

d) a carbonyl comprising unit having the formula:

—(CH₂)ₓCOR⁷

wherein R⁷ is:

i) —OH, in the case of carboxylic acids;

ii) —OR⁸, in the case of esters wherein R⁸ is hydrogen; C₁-C₁₅, preferably C₁-C₁₀, more preferably C₁-C₄ substituted or unsubstituted linear alkyl; C₃-C₁₅, preferably C₃-C₁₀, more preferably C₃-C₄ substituted or unsubstituted branched alkyl; C₂-C₂₂, preferably C₂-C₁₀, more preferably C₂-C₄ substituted or unsubstituted linear alkenyl; C₃-C₂₂ substituted or unsubstituted branched alkenyl, or mixtures thereof;

iii) —N(R⁹)₂ in the case of amides wherein each R⁹ is independently hydrogen; C₁-C₁₅, preferably C₁-C₁₀, more preferably C₁-C₄ substituted or unsubstituted linear alkyl; C₃-C₁₅, preferably C₃-C₁₀, more preferably C₃-C₄ substituted or unsubstituted branched alkyl; or mixtures thereof;

iv) C₁-C₂₂, preferably C₁-C₅ substituted or unsubstituted linear alkyl;

v) C₁-C₂₂, preferably C₃-C₅ substituted or unsubstituted branched alkyl;

vi) C₂-C₂₂, preferably C₂-C₅ substituted or unsubstituted linear alkenyl;

vii) C₃-C₂₂, preferably C₄-C₁₀ substituted or unsubstituted branched alkenyl;

viii) C₅-C₂₂, preferably C₆-C₁₀ substituted or unsubstituted cycloalkyl;

ix) C₆-C₂₂, preferably C₆-C₁₀ substituted or unsubstituted aryl;

x) C₆-C₂₂, preferably C₆-C₁₀ substituted or unsubstituted heterocyclicalkyl;

xi) C₆-C₂₂, preferably C₆-C₁₀ substituted or unsubstituted heterocyclicalkenyl;

the index is from 0 to 22;

e) alkyleneoxy units having the formula:

—(CR¹⁰R¹¹)_{y}(CHR¹²CHR¹³O)_{z}R¹⁴

wherein each R¹⁰, R¹¹, and R¹² is independently;

i) hydrogen;

ii) —OH;

iii) C₁-C₄ alkyl, preferably methyl;

iv) or mixtures thereof; preferably R¹⁰, R¹¹, and R¹² are each hydrogen;

R³ is:

i) hydrogen;

ii) C₁-C₄ alkyl, preferably methyl;

iii) or mixtures thereof; preferably R¹³ is methyl or hydrogen, more preferably hydrogen;

R¹⁴ is:

i) hydrogen;

ii) C₁-C₄ alkyl, preferably methyl;

iii) or mixtures thereof; preferred R¹⁴ is hydrogen;

R¹⁰ and R¹¹ can be taken together to form a C₃-C₆ spiroannulated ring, carbonyl unit, or mixtures thereof; y has the value from 0 to 10, z has the value from 1 to 50;

f) any two R², R³, R⁴, R⁵, or R⁶ units can be taken together, and where feasible, combined to form:

i) a carbonyl moiety;

ii) a C₃-C₆ spiroannulated ring;

iii) a heterocyclic aromatic ring comprising from 5 to 7 atoms;

iv) a non-heterocyclic aromatic ring comprising from 5 to 7 atoms;

v) a heterocyclic ring comprising from 5 to 7 atoms;

vi) a non-heterocyclic ring comprising from 5 to 7 atoms;

vii) or mixtures thereof;

g) and mixtures thereof;

the index y is an integer from 1 to 3, preferably 1 or 2, more preferably 1.

Fragrance Delivery System

The present invention further relates to fragrance delivery systems which are suitable for use in delivering an enhanced duration aesthetic benefit. The fragrance delivery systems are suitable for use and are compatible with any composition which may require a fragrance inter alia fine fragrances, perfumes, personal care products, deodorants, shampoos, laundry detergents, malodor masking agents.

In general, the compositions of the present invention, other than fine fragrances or perfumes which are described herein below, comprise from about 0.01%, preferably from about 0.05%, more preferably from about 0.1%, most preferably from about 0.5% to about 10%, preferably to about 7%, more preferably to about 5%, most preferably to about 3% by weight, of a fragrance delivery system. Preferably, the fragrance delivery systems comprise:

A) from about 1%, preferably from about 10%, more preferably from about 25% to about 100%, preferably to about 90%, more preferably to about 75%, most preferably to about 50% by weight, of a pro-fragrance component comprising:

i) at least 1% by weight (10 ppb of the composition which employs the fragrance delivery system), of an α-tertiary carbon comprising aldehyde releasing pro-fragrance component according to the present invention as described herein;

ii) optionally at least 1% by weight (10 ppb of the composition which employs the fragrance delivery system), of one or more pro-accords formed from at least one fragrance raw material, wherein said pro-accord is selected from the group consisting of acetals, ketals, orthoesters, orthocarbonates, and mixtures thereof, each pro-accord releasing upon hydrolysis said fragrance raw material from which it is formed, said fragrance raw materials selected from the group consisting of primary, secondary, and tertiary alcohols, aldehydes, ketones, esters, carbonates, and mixtures thereof, provided each pro-accord:

a) is formed from at least one fragrance raw material having a molecular weight greater than or equal to about 100 g/mol;

b) has a fragrance release half-life of greater than or equal to about 0.1 hours at pH 5.3 and less than or equal to about 12 hours at pH 2.5 when measured in NaH₂PO₄ buffer;

iii) the balance carriers, stabilizers, and other adjunct ingredients; and

B) optionally from about 1%, preferably from about 25% to about 99%, preferably to about 90%, more preferably to about 75%, most preferably to about 50% by weight, a fragrance raw material component comprising:

i) optionally at least 1% by weight, of a mixture of one or more base note fragrances;

ii) optionally at least 1% by weight, of a mixture of one or more top or middle note fragrances;

ii) optionally the balance carriers, fixatives, and other adjunct ingredients.

However, some compositions according to the present invention are fine fragrances or perfumes. These embodiments typically comprise only fragrance raw materials, pro-fragrances, pro-accords, carriers, and stabilizers. The fine fragrance and perfume compositions which utilize the cyclic pro-fragrances of the present invention comprise:

a) at least 0.01%, preferably from about 0.1%, more preferably from about 5%, most preferably from about 20% to about 100%, preferably to about 75%, more preferably to 50% by weight, of an α-tertiary carbon comprising aldehyde releasing pro-fragrance component according to the present invention;

b) optionally from about 1%, preferably from about 25% to about 99.99%, preferably to about 99.9%, more preferably to about 95%, most preferably to about 50% by weight, of one or more pro-accords which comprise n fragrance raw materials but which release n+1 fragrance raw materials, preferably orthoesters, orthocarbonates, β-ketoesters, and mixtures thereof;

c) optionally from about 1%, preferably from about 25% to about 99.99%, preferably to about 99.9%, more preferably to about 95%, most preferably to about 50% by weight, of one or more pro-fragrances selected from the group consisting of acetals, ketals, orthoesters, orthocarbonate, ortholactones, β-ketoesters, and mixtures thereof;

d) optionally from about 1%, preferably from about 25% to about 99.99%, preferably to about 99.9%, more preferably to about 95%, most preferably to about 50% by weight, of one or more fragrance raw materials, preferably one or more fragrance raw materials which are released by a pro-fragrance or pro-accord which comprises the fragrance delivery system; and

e) optionally one or more carriers, fixatives, stabilizers, or adjunct ingredients.

When present, the carriers, fixatives, or stabilizers will comprise the balance of the compositions. Typical carriers are methanol, ethanol (preferred), iso-propanol, polyethylene glycol, as well as water in some instances, especially as a vehicle to deliver materials which provide reserve alkalinity to the fragrance delivery system. Fixatives serve to lower the volatility of certain top and middle notes in order to extend their contact time on skin. Adjunct ingredients include perfume raw material components which are essential oils and are therefore not a single chemical entity. In addition, the adjunct ingredients may be mixtures of materials which serve a purpose in addition to providing a pleasurable odor (i.e., an astringent in a personal hygiene article).

For the purposes of the fragrance delivery systems of the present invention, a “pro-accord which comprises n fragrance raw materials but which releases n+1 fragrance raw materials” is defined as “a compound which is prepared from one or more fragrance raw materials, said fragrance raw material being chemically transformed into a “releasable form” such that when said releasable form breaks down, the original fragrance raw material is released as well as at least one other fragrance raw material which was not a starting material used in forming the releasable form”. Pro-accords of this type are suitably described in U.S. patent application Ser. No. 09/028,823 filed Feb. 24, 1998.

As described herein above, an optional component of the fragrance delivery systems of the present invention are pro-fragrances or pro-accords which are not heterocyclic aldehyde releasing pro-fragrances. The optional pro-accords or pro-fragrances are equally functional in either personal care compositions inter alia lotions, creams, deodorants or personal fragrance compositions inter alia fine fragrances, perfumes.

Preferred optional pro-accords and/or pro-fragrances include, but are not limited to, orthoesters, orthocarbonates, acetals, ketals, ortholactones, and β-ketoesters.

Non-limiting examples of optional orthoesters which are suitable for use in the fragrance delivery systems of the present invention include tris-geranyl orthoformate, tris(cis-3-hexen-1-yl) orthoformate, tris(phenylethyl) orthoformate, bis(citronellyl) ethyl orthoacetate, tris(citronellyl) orthoformate, tris(cis-6-nonenyl) orthoformate, tris(phenoxyethyl) orthoformate, tris(geranyl, neryl) orthoformate (70:30 geranyl:neryl), tris(9-decenyl) orthoformate, tris(3-methyl-5-phenylpentanyl) orthoformate, tris(6-methylheptan-2-yl) orthoformate, tris([4-(2,2,6-trimethyl-2-cyclohexen-1-yl)-3-buten-2-yl] orthoformate, tris [3-methyl-5-(2,2,3-trimethyl-3-cyclopenten-1-yl)-4-penten-2-yl] orthoformate, trismenthyl orthoformate, tris(4-isopropylcyclohexylethyl-2-yl) orthoformate, tris-(6,8-dimethylnonan-2-yl) orthoformate, tris-phenylethyl orthoacetate, tris(cis-3-hexen-1-yl) orthoacetate, tris(cis-6-nonenyl) orthoacetate, tris-citronellyl orthoacetate, bis(geranyl) benzyl orthoacetate, tris(geranyl) orthoacetate, tris(4-isopropylcyclohexylmethyl) orthoacetate, tris(benzyl) orthoacetate, tris(2,6-dimethyl-5-heptenyl) orthoacetate, bis(cis-3-hexen-1-yl) amyl orthoacetate, and neryl citronellyl ethyl orthobutyrate.

Non-limiting examples of optional orthocarbonates which are suitable for use in the fragrance delivery systems of the present invention include bis(ethyl) bis(geranyl) orthocarbonate, bis(ethyl) bis(phenylethyl) orthocarbonate, bis(ethyl) bis(cis-3-hexenyl) orthocarbonate, bis(ethyl) bis(citronellyl) orthocarbonate, bis(ethyl) bis(linalyl) orthocarbonate, bis(ethyl) bis(menthyl) orthocarbonate, bis(dodecyl) bis(geranyl) orthocarbonate, and bis(dodecyl) bis(phenylethyl) orthocarbonate.

Non-limiting examples of optional acetals which are suitable for use in the fragrance delivery systems of the present invention include bis(cis-3-hexenyl) vanillin, bis(geranyl) cinnamaldehyde acetal, bis(2-phenylethyl) anisaldehyde acetal, bis (citronellyl) cyclamen aldehyde acetal, and bis(citronellyl) citral acetal.

Non-limiting examples of optional ketals which are suitable for use in the fragrance delivery systems of the present invention include bis(linalyl) β-ionone ketal, bis(dihydromyrcenyl) α-damascone ketal, bis(linalyl) 6,7-dihydro-1,1,2,3,3-pentamethyl-4(5H)-indanone ketal, bis(dihydromyrcenyl) β-ionone ketal, and bis(citronellyl) cis-jasmone ketal.

Non-limiting examples of optional β-ketoesters which are suitable for use in the fragrance delivery systems of the present invention include 2,6-dimethyl-7-octen-2-yl 3-(4-methoxyphenyl)-3-oxo-propionate, 3,7-dimethyl-1,6-octadien-3-yl 3-(α-naphthyl)-3-oxo-propionate, 2,6-dimethyl-7-octen-2-yl 3-(4-methoxyphenyl)-3-oxo-propionate, cis 3-hexen-1-yl 3-(β-naphthyl)-3-oxo-propionate, 2,6-dimethyl-7-octen-2-yl 3-(nonanyl)-3-oxo-propionate, 2,6-dimethyl-7-octen-2-yl 3-oxo-butyrate, 3,7-dimethyl-1,6-octadien-3-yl 3-oxo-butyrate, 2,6-dimethyl-7-octen-2-yl 3-(β-naphthyl)-3-oxo-2-methylpropionate, 3,7-dimethyl-1,6-octadien-3-yl 3-(β-naphthyl)-3-oxo-2,2-dimethylpropionate, 3,7-dimethyl-1,6-octadien-3-yl 3-(β-naphthyl)-3-oxo-2-methylpropionate, 3,7-dimethyl-2,6-octadienyl 3-(β-naphthyl)-3-oxo-propionate, and 3,7-dimethyl-2,6-octadienyl 3-heptyl-3-oxo-propionate.

Fragrance Release Half-life

One aspect of the present invention which is a key element in providing the formulator with a method for determining the manner in which a pro-fragrance according to the present invention releases its fragrance raw material, is the measurement of the pro-fragrance “Fragrance Release Half-Life, (FRHL). The pro-fragrances useful in the personal care compositions of the present invention generally have a delayed release of final fragrance raw material in order to achieve the increased fragrance longevity benefits described herein. However, the pro-fragrances generally also deliver the fragrance raw materials during a time period useful to the formulator, for example, within a time period desirable to the consumer.

For the purposes of the present invention the pro-accords generally have a FRHL of less than or equal to 12 hours when measured in NaH₂PO₄ buffer at pH 2.5 and greater than or equal to 0.1 hour when measured in NaH₂PO₄ buffer at pH 5.3. The “Fragrance Release Half-life” is defined herein as follows.

Pro-fragrances deliver their corresponding mixture of fragrance raw materials or fragrance accords according to the equation:

Pro-Fragrance→Fragrance Raw Material

wherein the fragrance raw material which is released may be released as a single component or a multiple fragrance raw material accord.

The rate at which the fragrance is released is defined by the formula:

Rate=k[Pro-fragrance]

and can be further expressed by the formula:

$$- \frac{ⅆ [ Pro \text{-} fragrance ]}{ⅆ t} = k [ Pro \text{-} fragrance ]$$

wherein k is the release rate constant and [Pro-fragrance] is the concentration of pro-fragrance. For the purposes of the present invention the “Fragrance Release Half-life”, t_{1/2}, is related to the release rate constant by the formula:

$${t}_{1 / 2} = \frac{0.693}{k}$$

and this relationship is used for the purposes of the present invention to determine the “FRHL.

Due to the hydrophobic nature of some pro-accords, it is necessary to conduct the determination of t_{1/2} and k in a mixture of 90/10 dioxane/phosphate buffered water. The phosphate buffered water is prepared by admixing 3.95 mL of 85% phosphoric acid (H₃PO₄) and 24 g of sodium dihydrogen phosphate (NaH₂PO₄) with one liter of water. The pH of this solution is approximately 2.5. Next 10 mL of the phosphate buffer is admixed with 90 mL of dioxane and the pro-fragrance to be analyzed is added. The hydrolysis kinetics are then monitored by conventional HPLC at 30° C.

In some instances, it is desirable to formulate a fragrance delivery system having one or more pro-fragrances which deliver a rapid release of fragrance raw material in addition to the delayed onset of a fragrance. In such cases the hydrolysis rate, and therefore the determination of t_{1/2} must be measured in a buffer system which can accommodate this more rapid hydrolysis rate.

The pro-fragrances of the present invention are stable under pH conditions encountered in the formulation and storage of fine perfume, personal care and personal hygiene articles which have a pH of from about 7.1 to 11.5, and during solution-use of such products. Due to their high molecular weight and hydrophobicity, these pro-fragrances and/or pro-accords remain deposited upon skin even when exposed to water (i.e. when formulated into a sun screen). Because the pro-fragrances are subject to hydrolysis when the pH is reduced, they hydrolyze to release their component fragrance compounds when applied to skin or are exposed even to reduced pH such as present in air and humidity. The reduction in pH should be at least 0.1, preferably at least about 0.5 units. Preferably the pH is reduced by at least 0.5 units to a pH of 7.5 or less, more preferably 6.9 or less. Preferably, the solution in which the pro-accord is applied is alkaline.

Odor Value

The pro-fragrances of the present invention typically have an Odor Value greater than or equal to about 1, preferably greater than or equal to about 5, more preferably greater than or equal to about 10. The term “Odor Value” is defined by the following formula:

$$OV = \frac{[ Concentration of FRM ]}{ODT}$$

wherein OV is the odor value of the fragrance raw material released upon the skin by the pro-accord. The odor value is the concentration of the fragrance raw material, FRM, on the skin surface divided by the Odor Detection Threshold, ODT. The term “level of noticeability” is often applied to and/or substituted for the term “odor value”.

Odor Detection Threshold

For the purposes of the present invention the term “odor detection threshold” is defined as the level at which a fragrance raw material is perceptible to the average human. The odor detection threshold (ODT) of the compositions of the present invention are preferably measured by carefully controlled gas chromatograph (GC) conditions as described hereinbelow. The preferred fragrance raw materials of the present invention have an ODT of at least about 100 part per billion (ppb), more preferably 10 ppb, most preferably 1 ppb. Fragrance raw materials having an ODT greater than 10 parts per million (ppm) are typically avoided unless useful as an adjunct ingredient, for example, as an adjunct alcohol when adjusting the fragrance release half-life of an orthoester.

Determination of Odor Detection Thresholds is as follows. A gas chromatograph is characterized to determine the exact volume of material injected by a syringe, the precise split ratio, and the hydrocarbon response using a hydrocarbon standard of known concentration and chain-length distribution. The air flow rate in accurately measured and, assuming the duration of a human inhalation to last 0.02 minutes, the sampled volume is calculated. Since the precise concentration at the detector at any point in time is known, the mass per volume inhaled is known and hence the concentration of material. To determine whether a material has a threshold below 10 ppb, solutions are delivered to the sniff port at the back-calculated concentration. A panelist sniffs the GC effluent and identifies the retention time when odor is notice. The average over all panelists determines the threshold of noticeability or ODT. The necessary amount of analyte is injected onto the column to achieve a 10 ppb concentration at the detector. Typical gas chromatograph parameters for determining odor detection thresholds are listed below.

GC: 5890 Series H with FID detector 7673 Autosampler

Column: J&W Scientific DB-1, length 30 m, i.d. 0.25 mm, film thickness 1 μm.

Split Injection: 17/1 split ratio

Autosampler: 1.13 □l/injection

Column flow: 1.10 mL/min

Air flow: 345 mL/min

Inlet temperature: 245° C.

Detector temperature: 285° C.

Temperature Information:

Initial temperature: 50° C.

Rate: 5° C./min

Final temperature: 280° C.

Final time: 6 min

Leading assumptions: 0.02 minutes per sniff and that GC air adds to sample dilution.

Skin Performance Index

Although a pro-fragrance or pro-accord may comprise a fragrance release half-life which ensures delivery of a fragrance raw material during a period of time useful to the formulator, unless the fragrance raw materials which comprise said fragrance delivery system have ODT values large enough to be perceived by the user, the formulator will be compelled to use an inordinate amount of material to achieve a suitable fragrance level.

The pro-fragrances of the present invention have a Skin Performance Index (SPI) greater than or equal to 0.1, preferably greater than or equal to 0.5. The Skin Performance Index is defined by the following:

$$SPI = \frac{{[ Odor Value ]}^{*}}{{t}_{1 / 2}}$$

wherein the term [Odor Value]* is the estimated concentration of the fragrance raw material in the headspace above a solution of the fragrance raw material as measured in a 1% solution of ethanol, and t_{1/2} is the fragrance release half-life measured at pH 5.3 in the above described buffer. For the purposes of the present invention, the t_{1/2} of the SPI is measured at 5.3 and the value of the fragrance release half-life is preferably from 0.1 hours to 60 hours.

The [Odor Value]* is an estimation of the vapor pressure of the fragrance raw material using empirically determined KOVATS indices. “The Vapor Pressures of Pure Substances”, T. Boublik et al., Elseiver, N.Y. (1973) incorporated herein by reference, describes an index line for normal alkanes wherein C₁₀ is equal to 30,000 ppb, C₁₂ is equal to 3,000 ppb, C₁₄ is equal to 300 ppb, C₁₆ is equal to 30 ppb, etc. Using these values as reference standards, the KOVATS index of a fragrance raw material is obtained from gas chromatographic analysis of the FRM and the experimental index is then used to determine the relative vapor pressure and hence the head space concentration of the fragrance raw material.

“New Method for Estimating Vapor Pressure by the Use of Gas Chromatography” J. Chromatography A, 79 p 123-129, (1996) and “Simple and Versatile Injection System for Capillary Gas Chromatographic Columns: Performance Evaluation of a System Including Mass Spectrometric and Light Pipe Fourier-Transform Infrared Detection”, J. Chromatography A, 713, p 201-215, (1996) included herein by reference, further describe methods and techniques suitable for use in determining the vapor pressure and head space concentration of FRM's as they relate to the term [Odor Value]* of the present invention.

Using the criteria set forth in the present invention inter alia fragrance release half-life, odor value, odor detection threshold, skin performance index, the formulator is able to fashion an aldehyde or ketone releasing cyclic pro-fragrance. By manipulation of the R², R³, R⁴, R⁵ and R⁶ units of the cyclic pro-fragrances of the present invention, the release rate of either an aldehyde or ketone fragrance raw material can be adjusted. Several different pro-fragrances which release the same fragrance raw material, but at differing rates or levels, can be admixed to further prolong or extend the period of fragrance raw material delivery.

The following is a non-limiting example of a b-amino ketone pro-fragrance according to the present invention.

### EXAMPLE 1

### Oxazolidine of α-methyl melonal and DL-serine methyl ester

1. Synthesis of α-methyl melonal

To a solution of KH (1.2 mol) in anhydrous THF is added methyl iodide (1.2 mole). The solution is cooled to 0° C. and melonal (0.12 mole) dissolved in anhydrous THF is added over 40 minutes. Once the addition is complete, the solution is allowed to react for an additional 30 minutes. The reaction is quenched by the addition of water and the product isolated by extraction with diethyl ether. The oil which results after removal of the solvent is further purified by vacuum distillation.

2. Oxazolidine Formation

Racemic serine methyl ester hydrochloride (1.0 mol) is neutralized by the addition of sodium methoxide (1.1 mol) as a 25% solution in MeOH. After stirring about 15 minutes, anhydrous Na₂SO₄ (2 mol) is added followed by a -methyl melonal (1 mole). The solution is stirred for 24 hr, filtered through a bed of Celite 545 and concentrated under reduced pressure. The resulting residue is taken up in toluene, washed thrice with sodium bicarbonate and concentrated. The resulting oil is stirred 24 hr under a vacuum of 0.2 mm Hg to yield the desired product.

A personnel cleanser composition is prepared by combining the following ingredients using conventional mixing techniques.

TABLE I

Weight %

| Ingredients | 2 | 3 | 4 | 5 |
|---|---|---|---|---|
| Phase A | | | | |
| Water | QS 100 | QS 100 | QS 100 | QS 100 |
| Disodium EDTA | 0.100 | 0.100 | 0.100 | 0.100 |
| Glycerin | 4.00 | 4.00 | 4.00 | 4.00 |
| Methylparaben | 0.200 | 0.200 | 0.200 | 0.200 |
| C₁₀-C₃₀ alkyl | 0.150 | 0.150 | 0.150 | 0.150 |
| acrylate crosspolymer¹ | | | | |
| Carbomer 954² | 0.250 | 0.250 | 0.250 | 0.250 |
| Phase B | | | | |
| Stearic Acid | 0.110 | 0.110 | 0.110 | 0.110 |
| Stearyl alcohol | 0.875 | 0.875 | 0.875 | 0.875 |
| Cetyl alcohol | 0.875 | 0.875 | 0.875 | 0.875 |
| Propylparaben | 0.150 | 0.150 | 0.150 | 0.150 |
| Steareth-2 | — | 0.25 | 0.25 | 0.25 |
| Steareth-21 | — | 0.50 | 0.50 | 0.50 |
| Phase C | | | | |
| Sodium hydroxide³ | 0.130 | 0.130 | 0.130 | 0.130 |
| Phase D | | | | |
| Diisopropyl sebacate | 1.50 | 1.50 | 1.50 | 1.50 |
| Isohexadecane | 5.00 | 2.00 | 5.00 | 5.00 |
| Mineral Oil⁴ | — | 5.00 | — | — |
| Phase E | | | | |
| Phenoxyethanol | 0.5 | 0.5 | — | 0.5 |
| Fragrance delivery system⁵ | 1.5 | 1.5 | 2.20 | 1.5 |
| Glucose amide | 0.96 | 0.96 | 0.96 | 0.96 |

¹Available as Pemulen ® from B. F. Goodrich Corporation.
²Available as Carbomer ® 954 from B. F. Goodrich Corporation.
³As a 50% aqueous solution.
⁴Light mineral oil available as Drakeol 5 from Penreco, Dickenson, TX.
⁵A fragrance delivery system which comprises about 5% by weight of 2-(1,1,5-trimethylhex-4-enyl)-5-carboxymethyloxazolidine according to Example 1.

The above Examples 2-5 can be suitably prepared as follows. In a suitable vessel, the Phase A ingredients are mixed at room temperature to form a dispersion and heated with stirring to 70-80° C. In a separate vessel, the Phase B ingredients are heated with stirring to 70-80° C. Phase B is then added to Phase A with mixing to form the emulsion. Next, Phase C is added to neutralize the composition. The Phase D ingredients are added with mixing, followed by cooling to 45-50° C. The Phase E ingredients are then added with stirring, followed by cooling to 40° C. Phase F is heated with mixing to 40° C. and added to the emulsion, which is cooled to room temperature. The resulting cleansing composition is useful for cleansing the skin. The emulsion de-emulsifies upon contact with the skin.

What is claimed is:

1. A fragrance raw material having the formula: 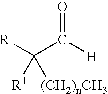
wherein R and R¹ are each independently:
a) C₁-C₁₀ substituted or unsubstituted linear alkyl;
b) C₃-C₁₅ substituted or unsubstituted branched alkyl;
c) C₂-C₁₅ substituted or unsubstituted linear alkenyl;
d) C₃-C₁₅ substituted or unsubstituted branched alkenyl;
e) C₃-C₁₅ substituted or unsubstituted cycloalkyl;
f) C₄-C₁₅ substituted or unsubstituted branched cycloalkyl;
g) C₄-C₁₅ substituted or unsubstituted cycloalkenyl;
h) C₅-C₁₅ substituted or unsubstituted branched cycloalkenyl;
i) C₆-C₁₅ substituted or unsubstituted aryl;
j) C₇-C₂₂ substituted or unsubstituted alkylenearyl;
k) C₇-C₂₂ substituted or unsubstituted arylenealkyl;
l) C₆-C₂₂ substituted or unsubstituted heterocyclicalkyl;
m) C₆-C₂₂ substituted or unsubstituted heterocyclicalkenyl;
n) hydroxyl;
o) nitrilo;
p) a carbonyl comprising unit having the formula:
—(CH₂)ₓCOR²
wherein R² is:
i) —OH;
ii) —OR³ wherein R³ is hydrogen, C₁-C₁₅ substituted or unsubstituted linear alkyl, C₁-C₁₅ substituted or unsubstituted branched alkyl, C₂-C₂₂ substituted or unsubstituted linear alkenyl, C₃-C₂₂ substituted or unsubstituted branched alkenyl, or mixtures thereof;
iii) —N(R⁴)₂ wherein R⁴ is hydrogen, C₁-C₆ substituted or unsubstituted linear alkyl, C₃-C₆ substituted or unsubstituted branched alkyl, or mixtures thereof;
iv) C₁-C₂₂ substituted or unsubstituted linear alkyl;
v) C₁-C₂₂ substituted or unsubstituted branched alkyl;
vi) C₂-C₂₂ substituted or unsubstituted linear alkenyl;
vii) C₃-C₂₂ substituted or unsubstituted branched alkenyl;
viii) C₃-C₂₂ substituted or unsubstituted cycloalkyl;
ix) C₆-C₂₂ substituted or unsubstituted aryl;
x) C₆-C₂₂ substituted or unsubstituted heterocyclicalkyl;
xi) C₆-C₂₂ substituted or unsubstituted heterocyclicalkenyl;
the index x is from 0 to 22;
q) alkyleneoxy units having the formula:
—(CR⁵R⁶)_{y}(CHR⁷CHR⁸O)_{z}R⁹
wherein each R⁵, R⁶, and R⁷ is independently;
i) hydrogen;
ii) —OH;
iii) C₁-C₄ alkyl;
iv) or mixtures thereof;
R⁸ is:
i) hydrogen;
ii) C₁-C₄ alkyl;
iii) or mixtures thereof;
R⁹ is:
i) hydrogen;
ii) C₁-C₄ alkyl;
iii) or mixtures thereof;
R⁵ and R⁶ can be taken together to form a C₃-C₆ spiroannulated ring, carbonyl unit, or mixtures thereof; y has the value from 0 to 10, z has the value from 1 to 50;
r) R and R¹ can be taken together to form:
i) a C₃-C₆ substituted or unsubstituted spiroannulated ring;
ii) a substituted or unsubstituted non-aromatic ring comprising from 5 to 7 atoms in the ring;
iii) a substituted or unsubstituted non-aromatic heterocyclic ring comprising from 5 to 7 atoms in the ring;
iv) a substituted or unsubstituted fused ring system comprising from 5 to 20 atoms; and
n is 0 or 1.

2. A fragrance delivery system comprising:
A) from about 1% by weight, of a pro-fragrance component, said pro-fragrance component comprising:
i) at least 1% by weight, of an □-tertiary carbon comprising aldehyde releasing pro-fragrance component having the formula: 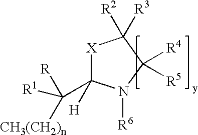
wherein X is oxygen or sulfur, n is 0 or 1, R and R¹ are each independently:
a) C₁-C₁₀ substituted or unsubstituted linear alkyl;
b) C₃-C₁₅ substituted or unsubstituted branched alkyl;
c) C₂-C₁₅ substituted or unsubstituted linear alkenyl;
d) C₃-C₁₅ substituted or unsubstituted branched alkenyl;
e) C₃-C₁₅ substituted or unsubstituted cycloalkyl;
f) C₄-C₁₅ substituted or unsubstituted branched cycloalkyl;
g) C₄-C₁₅ substituted or unsubstituted cycloalkenyl;
h) C₅-C₁₅ substituted or unsubstituted branched cycloalkenyl;
i) C₆-C₁₅ substituted or unsubstituted aryl;
j) C₇-C₂₂ substituted or unsubstituted alkylenearyl;
k) C₇-C₂₂ substituted or unsubstituted arylenealkyl;
l) C₆-C₂₂ substituted or unsubstituted heterocyclicalkyl;
m) C₆-C₂₂ substituted or unsubstituted heterocyclicalkenyl;
n) hydroxyl;
o) nitrilo;
p) a carbonyl comprising unit having the formula:
—(CH₂)ₓCOR²
wherein R² is:
i) —OH;
ii) —OR³ wherein R³ is hydrogen, C₁-C₁₅ substituted or unsubstituted linear alkyl, C₁-C₁₅ substituted or unsubstituted branched alkyl, C₂-C₂₂ substituted or unsubstituted linear alkenyl, C₃-C₂₂ substituted or unsubstituted branched alkenyl, or mixtures thereof;
iii) —N(R⁴)₂ wherein R⁴ is hydrogen, C₁-C₆ substituted or unsubstituted linear alkyl, C₃-C₆ substituted or unsubstituted branched alkyl, or mixtures thereof;
iv) C₁-C₂₂ substituted or unsubstituted linear alkyl;
v) C₁-C₂₂ substituted or unsubstituted branched alkyl;
vi) C₂-C₂₂ substituted or unsubstituted linear alkenyl;
vii) C₃-C₂₂ substituted or unsubstituted branched alkenyl;
viii) C₃-C₂₂ substituted or unsubstituted cycloalkyl;
ix) C₆-C₂₂ substituted or unsubstituted aryl;
x) C₆-C₂₂ substituted or unsubstituted heterocyclicalkyl;
xi) C₆-C₂₂ substituted or unsubstituted heterocyclicalkenyl;
the index x is from 0 to 22;
q) alkyleneoxy units having the formula:
—(CR⁵R⁶)_{y}(CHR⁷CHR⁸O)_{z}R⁹
wherein each R⁵, R⁶, and R⁷ is independently;
i) hydrogen;
ii) —OH;
iii) C₁-C₄ alkyl;
iv) or mixtures thereof;
R⁸ is:
i) hydrogen;
ii) C₁-C₄ alkyl;
iii) or mixtures thereof;
R⁹ is:
i) hydrogen;
ii) C₁-C₄ alkyl;
iii) or mixtures thereof;
R⁵ and R⁶ can be taken together to form a C₃-C₆ spiroannulated ring, carbonyl unit, or mixtures thereof; y has the value from 0 to 10, z has the value from 1 to 50;
r) R and R¹ can be taken together to form:
i) a C₃-C₆ substituted or unsubstituted spiroannulated ring;
ii) a substituted or unsubstituted non-aromatic ring comprising from 5 to 7 atoms in the ring;
iii) a substituted or unsubstituted non-aromatic heterocyclic ring comprising from 5 to 7 atoms in the ring;
each R², R³, R⁶ and each R⁴ and R⁵ pair are independently:
a) hydrogen;
b) R;
c) hydroxyl;
d) a carbonyl comprising unit having the formula:
—(CH₂)ₓCOR⁷
wherein R⁷ is:
i) —OH, in the case of carboxylic acids;
ii) —OR⁸ wherein R⁸ is C₁-C₁₅ substituted or unsubstituted branched alkyl; C₂-C₂₂ substituted or unsubstituted linear alkenyl; C₃-C₂₂ substituted or unsubstituted branched alkenyl, or mixtures thereof;
iii) —N(R⁹)₂ wherein each R⁹ is independently hydrogen; C₁-C₁₅ substituted or unsubstituted linear alkyl; C₃-C₁₅ substituted or unsubstituted branched alkyl; or mixtures thereof;
iv) C₁-C₂₂ substituted or unsubstituted linear alkyl;
v) C₁-C₂₂ substituted or unsubstituted branched alkyl;
vi) C₂-C₂₂ substituted or unsubstituted linear alkenyl;
vii) C₃-C₂₂ substituted or unsubstituted branched alkenyl;
viii) C₅-C₂₂ substituted or unsubstituted cycloalkyl;
ix) C₆-C₂₂ substituted or unsubstituted aryl;
x) C₆-C₂₂ substituted or unsubstituted heterocyclicalkyl;
xi) C₆-C₂₂ substituted or unsubstituted heterocyclicalkenyl;
the index x is from 0 to 22;
e) alkyleneoxy units having the formula:
—(CR¹⁰R¹¹)_{y}(CHR¹²CHR¹³O)_{z}R¹⁴
wherein each R¹⁰, R¹¹, and R¹² is independently;
i) hydrogen;
ii) —OH;
iii) C₁-C₄ alkyl;
iv) or mixtures thereof;
R¹³ is:
i) hydrogen;
ii) C₁-C₄ alkyl;
iii) or mixtures thereof;
R¹⁴ is:
i) hydrogen;
ii) C₁-C₄ alkyl;
iii) or mixtures thereof;
R¹⁰ and R¹¹ can be taken together to form a C₃-C₆ spiroannulated ring, carbonyl unit, or mixtures thereof; y has the value from 0 to 10, z has the value from 1 to 50;
f) any two R², R³, R⁴, R⁵, or R⁶ units can be taken together to form:
i) a carbonyl moiety;
ii) a C₃-C₆ spiroannulated ring;
iii) a heterocyclic aromatic ring comprising from 5 to 7 atoms;
iv) a non-heterocyclic aromatic ring comprising from 5 to 7 atoms;
v) a heterocyclic ring comprising from 5 to 7 atoms;
vi) a non-heterocyclic ring comprising from 5 to 7 atoms;
vii) or mixtures thereof;
g) and mixtures thereof;
the index y is an integer from 1 to 3;
ii) optionally at least 1% by weight, of one or more pro-accords formed from at least one fragrance raw material, wherein said pro-accord is selected from the group consisting of acetals, ketals, orthoesters, orthocarbonates, and mixtures thereof, each pro-accord releasing upon hydrolysis said fragrance raw material from which it is formed, said fragrance raw materials selected from the group consisting of primary, secondary, and tertiary alcohols, aldehydes, ketones, esters, carbonates, and mixtures thereof, provided each pro-accord:
a) is formed from at least one fragrance raw material having a molecular weight greater than or equal to about 100 g/mol;
b) has a fragrance release half-life of greater than or equal to about 0.1 hours at pH 5.3 and less than or equal to about 12 hours at pH 2.5 when measured in NaH₂PO₄ buffer;
iii) the balance carriers, stabilizers, and other adjunct ingredients; and
B) optionally from about 1% by weight, a fragrance raw material component comprising:
i) optionally at least 1% by weight, of a mixture of one or more base note fragrances;
ii) optionally at least 1% by weight, of a mixture of one or more top or middle note fragrances;
iii) optionally at least 1% by weight, of an a -tertiary carbon comprising aldehyde;
iv) optionally the balance carriers, fixatives, and other adjunct ingredients.

3. A system according to claim 2 comprising at least one pro-accord selected from the group consisting of tris-geranyl orthoformate, tris(cis-3-hexen-1-yl) orthoformate, tris(phenylethyl) orthoformate, bis(citronellyl) ethyl orthoacetate, tris(citronellyl) orthoformate, tris(cis-6-nonenyl) orthoformate, tris(phenoxyethyl) orthoformate, tris(geranyl, neryl) orthoformate (70:30 geranyl:neryl), tris(9-decenyl) orthoformate, tris(3-methyl-5-phenylpentanyl) orthoformate, tris(6-methylheptan-2-yl) orthoformate, tris([4-(2,2,6-trimethyl-2-cyclohexen-1-yl)-3-buten-2-yl] orthoformate, tris[3-methyl-5-(2,2,3-trimethyl-3-cyclopenten-1-yl)-4-penten-2-yl] orthoformate, trismenthyl orthoformate, tris(4-isopropylcyclo-hexylethyl-2-yl) orthoformate, tris-(6,8-dimethylnonan-2-yl) orthoformate, tris-phenylethyl orthoacetate, tris(cis-3-hexen-1-yl) orthoacetate, tris(cis-6-nonenyl) orthoacetate, tris-citronellyl orthoacetate, bis(geranyl) benzyl orthoacetate, tris(geranyl) orthoacetate, tris(4-isopropylcyclohexylmethyl) orthoacetate, tris(benzyl) orthoacetate, tris(2,6-dimethyl-5-heptenyl) orthoacetate, bis(cis-3-hexen-1-yl) amyl orthoacetate, and neryl citronellyl ethyl orthobutyrate.

4. A perfume or fine fragrance comprising:
A) from about 1% by weight, of a pro-fragrance delivery system, said pro-fragrance delivery system comprising:
i) at least 1% by weight, of an a -tertiary carbon comprising aldehyde releasing pro-fragrance component having the formula: 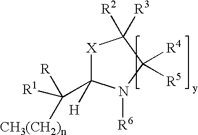
wherein X is oxygen or sulfur, n is 0 or 1, R and R¹ are each independently:
a) C₁-C₁₀ substituted or unsubstituted linear alkyl;
b) C₃-C₁₅ substituted or unsubstituted branched alkyl;
c) C₂-C₁₅ substituted or unsubstituted linear alkenyl;
d) C₃-C₁₅ substituted or unsubstituted branched alkenyl;
e) C₃-C₁₅ substituted or unsubstituted cycloalkyl;
f) C₄-C₁₅ substituted or unsubstituted branched cycloalkyl;
g) C₄-C₁₅ substituted or unsubstituted cycloalkenyl;
h) C₅-C₁₅ substituted or unsubstituted branched cycloalkenyl;
i) C₆-C₁₅ substituted or unsubstituted aryl;
j) C₇-C₂₂ substituted or unsubstituted alkylenearyl;
k) C₇-C₂₂ substituted or unsubstituted arylenealkyl;
l) C₆-C₂₂ substituted or unsubstituted heterocyclicalkyl;
m) C₆-C₂₂ substituted or unsubstituted heterocyclicalkenyl;
n) hydroxyl;
o) nitrilo;
p) a carbonyl comprising unit having the formula:
—(CH₂)ₓCOR²
wherein R² is:
i) —OH;
ii) —OR³ wherein R³is hydrogen, C₁-C₁₅ substituted or unsubstituted linear alkyl, C₁-C₁₅ substituted or unsubstituted branched alkyl, C₂-C₂₂ substituted or unsubstituted linear alkenyl, C₃-C₂₂ substituted or unsubstituted branched alkenyl, or mixtures thereof;
iii) —N(R⁴)₂ wherein R⁴is hydrogen, C₁-C₆ substituted or unsubstituted linear alkyl, C₃-C₆ substituted or unsubstituted branched alkyl, or mixtures thereof;
iv) C₁-C₂₂ substituted or unsubstituted linear alkyl;
v) C₁-C₂₂ substituted or unsubstituted branched alkyl;
vi) C₂-C₂₂ substituted or unsubstituted linear alkenyl;
vii) C₃-C₂₂ substituted or unsubstituted branched alkenyl;
viii) C₃-C₂₂ substituted or unsubstituted cycloalkyl;
ix) C₆-C₂₂ substituted or unsubstituted aryl;
x) C₆-C₂₂ substituted or unsubstituted heterocyclicalkyl;
xi) C₆-C₂₂ substituted or unsubstituted heterocyclicalkenyl;
the index x is from 0 to 22;
q) alkyleneoxy units having the formula:
—(CR⁵R⁶)_{y}(CHR⁷CHR⁸O)_{z}R⁹
wherein each R⁵, R⁶, and R⁷ is independently;
i) hydrogen;
ii) —OH;
iii) C₁-C₄ alkyl;
iv) or mixtures thereof;
R⁸ is:
i) hydrogen;
ii) C₁-C₄ alkyl;
iii) or mixtures thereof;
R⁹ is:
i) hydrogen;
ii) C₁-C₄ alkyl;
iii) or mixtures thereof;
R⁵ and R⁶ can be taken together to form a C₃-C₆ spiroannulated ring, carbonyl unit, or mixtures thereof; y has the value from 0 to 10, z has the value from 1 to 50;
r) R and R¹ can be taken together to form:
i) a C₃-C₆ substituted or unsubstituted spiroannulated ring;
ii) a substituted or unsubstituted non-aromatic ring comprising from 5 to 7 atoms in the ring;
iii) a substituted or unsubstituted non-aromatic heterocyclic ring comprising from 5 to 7 atoms in the ring;
iv) a substituted or unsubstituted fused ring system comprising from 5 to 20 atoms;
each R², R³, R⁶ and each R⁴ and R⁵ pair are independently:
a) hydrogen;
b) R;
c) hydroxyl;
d) a carbonyl comprising unit having the formula:
—(CH₂)ₓCOR⁷
wherein R⁷ is:
i) —OH, in the case of carboxylic acids;
ii) —OR⁸ wherein R⁸ is C₁-C₁₅ substituted or unsubstituted branched alkyl; C₂-C₂₂ substituted or unsubstituted linear alkenyl; C₃-C₂₂ substituted or unsubstituted branched alkenyl, or mixtures thereof;
iii) —N(R⁹)₂ wherein each R⁹ is independently hydrogen; C₁-C₁₅ substituted or unsubstituted linear alkyl; C₃-C₁₅ substituted or unsubstituted branched alkyl; or mixtures thereof;
iv) C₁-C₂₂ substituted or unsubstituted linear alkyl;
v) C₁-C₂₂ substituted or unsubstituted branched alkyl;
vi) C₂-C₂₂ substituted or unsubstituted linear alkenyl;
vii) C₃-C₂₂ substituted or unsubstituted branched alkenyl;
viii) C₅-C₂₂ substituted or unsubstituted cycloalkyl;
ix) C₆-C₂₂ substituted or unsubstituted aryl;
x) C₆-C₂₂ substituted or unsubstituted heterocyclicalkyl;
xi) C₆-C₂₂ substituted or unsubstituted heterocyclicalkenyl;
the index x is from 0 to 22;
e) alkyleneoxy units having the formula:
—(CR¹⁰OR¹¹)_{y}(CHR¹²CHR¹³O)_{z}R¹⁴
wherein each R¹⁰, R¹¹, and R¹² is independently;
i) hydrogen;
ii) —OH;
iii) C₁-C₄ alkyl;
iv) or mixtures thereof;
R¹³ is:
i) hydrogen;
ii) C₁-C₄ alkyl;
iii) or mixtures thereof;
R¹⁴ is:
i) hydrogen;
ii) C₁-C₄ alkyl;
iii) or mixtures thereof;
R¹⁰ and R¹¹ can be taken together to form a C₃-C₆ spiroannulated ring, carbonyl unit, or mixtures thereof; y has the value from 0 to 10, z has the value from 1 to 50;
f) any two R², R³, R⁴, R⁵, or R⁶ units can be taken together to form:
i) a carbonyl moiety;
ii) a C₃-C₆ spiroannulated ring;
iii) a heterocyclic aromatic ring comprising from 5 to 7 atoms;
iv) a non-heterocyclic aromatic ring comprising from 5 to 7 atoms;
v) a heterocyclic ring comprising from 5 to 7 atoms;
vi) a non-heterocyclic ring comprising from 5 to 7 atoms;
vii) or mixtures thereof;
g) and mixtures thereof;
the index y is an integer from 1 to 3;
ii) optionally at least 1% by weight, of one or more pro-accords formed from at least one fragrance raw material, wherein said pro-accord is selected from the group consisting of acetals, ketals, orthoesters, orthocarbonates, and mixtures thereof, each pro-accord releasing upon hydrolysis said fragrance raw material from which it is formed, said fragrance raw materials selected from the group consisting of primary, secondary, and tertiary alcohols, aldehydes, ketones, esters, carbonates, and mixtures thereof, provided each pro-accord:
a) is formed from at least one fragrance raw material having a molecular weight greater than or equal to about 100 g/mol;
b) has a fragrance release half-life of greater than or equal to about 0.1 hours at pH 5.3 and less than or equal to about 12 hours at pH 2.5 when measured in NaH₂PO₄ buffer;
iii) the balance carriers, stabilizers, and other adjunct ingredients;
B) from about 1% by weight, a fragrance raw material component comprising one or more fragrance raw material selected from the group consisting of aldehydes, ketones, alcohols, ethers, esters, nitro compounds, cyclic and acyclic hydrocarbyl fragrances, unsaturated hydrocarbyl fragrances, and mixtures thereof; and
C) the balance fixatives, carriers, and adjunct ingredients.
